(19) United States
(12) Patent Application Publication
(10) Pub. No.: US 2008/0063633 A1
(43) Pub. Date: Mar. 13, 2008

(54) Obtaining and Use of Therapeutic Antibodies Entering Into the Cell

(75) Inventors: Andres Valkna, Tallinn (EE); Pritt Kogerman, Tallinn (EE)

Correspondence Address: BIRCH STEWART KOLASCH & BIRCH, PO BOX 747, FALLS CHURCH, VA, 22040-0747, US

(73) Assignee: ROBERT BOSCH GMBH, STUTTGART GERMANY (DE)

(21) Appl. No.: 10/528,073

(22) PCT Filed: Sep. 16, 2003
(86) PCT No.: PCT/EE03/00005

§ 371 Date: Mar. 17, 2005

(30) Foreign Application Priority Data
Sep. 17, 2002 (EE) P200200531

(51) Int. Cl. C07K 16/00; A61K 39/395; A61P 35/00

(52) U.S. Cl. 424/130.1; 530/387.1

(57) ABSTRACT

The invention relates to the development of a novel technology of (cancer-) specific antibodies entering into the cell and their use for treatment of human diseases (primarily cancer). Such antibody (drug) would act by directly modulating the cancer-generating signals. The expected effects and principles of action of such antibodies are inactivation of intracellular proteins and thus they could be used for the treatment of diseases, where the activity of intracellular proteins must be modulated for effective treatment (primarily malignant tumors, but also many other diseases, which can be treated by inactivation of intracellular proteins). Above-mentioned antibody technology would also be applied elsewhere (e.g. scientific laboratories, that are engaged in investigation of intracellular proteins etc).

### TECHNICAL FIELD

The invention relates to the use of therapeutic antibodies entering into the cell. Such therapeutic antibodies would be used for the treatment of cancer and for other applications.

### BACKGROUND ART

All fundamental biological processes, including development, immunity and tumorigenesis, are related to the selective and differential expression of genes in different tissues and cell types. The formation of many malignant tumors has shown to be caused by changes in signals responsible of selective expression of genes. One of the goals of modem molecular medicine is to find the ways of regulating the expression of genes in living organisms and developing new treatment strategies on their basis.

Cancer is the second frequent cause of death in the developed world, thus the keen interest in the molecular mechanisms of the formation of malignant tumors and efficient treatment thereof. The application of complex methods of treatment (combining surgery, radiotherapy and cytostatic treatment) have considerably extended the lifespan of patients and improved their quality of life, however, no major breakthrough has been achieved to date. This is why especially the last decade has seen active research into the possibilities of using principally new methods of treatment (biological treatment, gene therapy etc.).

### DISCLOSURE OF INVENTION

The limited number of disease-specific cell surface markers is the main problem in immunotherapy. A number of intracellular disease-related molecules have been established and described in detail; unfortunately these are not accessible to the conventional antibodies. Our technology enables the use of these intracellular targets and thus to considerably increase the number of specific markers accessible to antibodies. The modified antibodies used by us are not toxic, nor are the component parts used for obtaining such antibodies toxic.

The subject of this invention is a technology for novel (cancer)-specific antibodies entering into the cell. Such antibodies would act by directly modulating the cancer-specific signals. The expected effects and principles of action of such antibodies are inactivation of intracellular proteins, thus these could be used for the treatment of diseases, where the activity of intracellular proteins must be modulated for effective treatment (primarily malignant tumors and diseases, which can be treated by inactivation of intracellular immunogenic therapeutic targets (proteins, glycoproteins etc.)). The above-mentioned antibody technology would also be applied elsewhere (e.g. scientific laboratories that are engaged in investigation of intracellular proteins etc.).

### Short Overview of Technologies

### Antibodies

Antibodies are proteins, naturally produced by the immune system as part of the immune response to foreign substances (antigens). Antibodies can be produced against molecule of interest, by using these molecules (or parts of these molecules) as antigens. In biomedicine antibodies that recognise specifically cell surface elements like membrane proteins (receptors) and non-protein components are of special interest as potential drugs. Since these surface markers can be cell type specific, thus it is possible to generate antibodies, which only recognise specific cell type. This feature can be used in the treatment of various diseases: inflammatory diseases, autoimmune diseases (allergic responses), conditions related to the transplantation of tissues, cardiovascular diseases, infectious diseases and primarily various tumors. However, the list should not be seen as an all-inclusive. In the conventional sense antibody therapy does mean the use of antibodies recognising the molecules or cell types causing previously named diseases and conditions.

The technology of generating antibodies is very well described whereas two different strategies exist—monoclonal antibody technology and polyclonal antibody technology. Both types of antibodies are widely used for different research and development purposes. However, such standard antibodies do not have significant potential as therapeutic agents due to their high molecular mass, their inability to enter the cells, their insufficient efficacy of diffusion into the tissues for effective treatment. In addition, standard antibodies are relatively unstable and in some cases non-specific side effects have been observed as well.

Antibodies have been recently expressed intracellularly in several systems to neutralise the function of endogenous target proteins (Ridder et al., 1995). These attempts to use “intrabodies” were made in general by transfection of scFv (single chain fragments variable) expressing vectors, and led to a number of promising results in several fields: cancer, Huntington's disease, viral diseases (Lecerf et al., 2001; Steinberger et al., 2000; Strube and Chen, 2002). These achievements showed that intracellular expression of antibodies or scFv could efficiently target some proteins and modify the cell's biology. scFvs—having the VH and the VL domains bridged by a linker peptide—represent the minimal intact binding species of an antibody, and seem to have similar functions as corresponding Fabs. However, the activity of such scFv molecules is greatly limited by their instability and folding efficiency in the reducing intracellular environment. Also, the stability of scFv seems to be dramatically increased when they are engineered as scFvFc (Strube and Chen, 2002). Finally, the delivery of the scFv “intrabodies” remains a problematic issue for their potential therapeutic applications.

We have developed a strategy for resolving these problems; in this patent application we prove the possibilities of implementing these strategies for treatment purposes and other potential applications. We have used the advantage of recent progress in peptide-mediated membrane penetration to directly and efficiently deliver the antibodies or the scFvs to their intracellular target proteins, avoiding the problems of intracellular misfolding in the cellular environment.

### Cell Penetrating Peptides

The use of peptide vector molecules (cell-penetrating peptides, CPPs) for transporting biologically active molecules has several advantages. It has proved effective in all eukaryotic cells tested so far, which allows them to be used en masse. The most recent results permit them also to be used as vectors penetrating the blood-brain barrier (Schwarze et al., 1999).

Transportan is a peptide in which the fragments of the neuropeptide galanin and of mastoparan, a toxic amphiphilic peptide found in wasp venom, are combined. It has been demonstrated that Transportan penetrates cell membranes using nonenzymatic mechanism. After penetration Transportan localises to the cell nucleus, where it further colocalises with nucleoli. It has been demonstrated both in vitro and in vivo that various molecules that naturally do not enter the cell (e.g. proteins, peptides, peptide nucleic acids (PNA)) will localise to the nucleus if coupled to transportan (Pooga et al., 1998). Thus Transportan is a suitable transport peptide for transporting proteins, also including antibodies into the cell. Transportan does not cause significant cytotoxic effects. To date a large number of other peptides able to enter the cell have been described, thus our invention can use any such cell penetrating peptide, which is understandable to a person skilled in the art. (Cell Penetrating Peptide; Advances and Applications, Editor Ü. Langel, CRC Press 22).

### Skin Cancer and GLI Proteins

Skin cancer (both melanomas and forms of non-melanoma skin cancer) is the most common type of cancer in the Western world. Non-melanoma skin cancer has become the second-most common type of skin cancer in women, with its incidence having doubled during the past decade. The main risk factor in both is the ultraviolet radiation. The Basal Cell Carcinoma (BCC) is the most widely spread malignant tumor in the Western world. BCCs can often be treated surgically, therefore the most important result of having a drug to treat this type of tumor would be the increase of cost effectiveness of treatment achieved by avoiding the need for surgery.

It has been shown that a large number of both hereditary and sporadic cases of BCC have been caused by mutations in the PTCH gene (human homologue of Drosophila Patched) encoding the receptor of the Sonic hedgehog (Shh) factor that leads to alterations in the signalling pathway mediated by this receptor (Dahmane et al., 1997). The mechanism behind the signalling pathway described above is to control the positioning of cells during embryogenesis. GLI proteins are transcriptor factors that act as effectors of this signalling pathway. GLI proteins interact in the cytoplasm with the protein of the tumor suppressor gene SUFUH (Kogerman et al., 1999) resulting in inactivation of the GLI protein.

### Pharmaceutical Composition

The present invention is also directed to a pharmaceutical composition, comprising the molecules of the invention in association with pharmaceutically acceptable carriers and additives. Such pharmaceutical composition can be obtained by applying methods and standard materials used in pharmaceutical practice.

Moreover, the present invention is also directed to a method for the treatment of a disease or health disorder in humans or animals. Such method comprises the administration of a pharmaceutically acceptable dose of the invented molecule to humans or animals.

The above-mentioned pharmaceutical composition can be administered orally, intravenously or intraperitoneally. The preferred route of administration is intravenous.

### BRIEF DESCRIPTION OF THE FIGURES

FIG. 1 shows the Transportan TP-10 HPLC chromatogram (FIG. 1A) and the MALDI-TOF spectrum (FIG. 1B).

FIG. 2 shows the mouse antiGLI IgG-Transportan TP10 conjugate (FIG. 2A), the mouse FITC-conjugated anti-IgG (FIG. 2B), the mouse anti-GLI1 (IgG)-Transportan TP10 conjugate (FIG. 2C), the mouse FITC-conjugated anti-IgG (FIG. 2D).

FIG. 3A shows the production and purification of the cell penetrating recombinant protein. The figure shows the image of Coomassie brilliant blue-stained SDS-polyacryl-amide gel. Lane 1: molecular weight marker. Lane 2: uninduced E. coli cell lysate; Lanes 3 and 4: cell lysate, where the expression of the construct has been induced by IPTG. Lanes 5-8: protein fractions 1-4 eluted from glutathione-agarose.

FIG. 3B shows the internalisation of the recombinant protein into human 293 cells. The cells were incubated with recombinant proteins and fluorescent anti-GST antibodies (upper image) detected their internalisation into the cells. The image below depicts the phase-contrast image of the same field.

### DETAILED DESCRIPTION OF THE INVENTION

We have produced monoclonal antibodies against GLI1 and GLI3 proteins. We have conducted preliminary studies and demonstrated that antibodies coupled with cell penetrating transport peptides, are able to effectively penetrate the cell membranes and that the coupling of such peptides to antibodies does not reduce the ability of the antibodies to recognise specific antigens.

### EXAMPLE 1

### Obtaining and Characterisation of Polyclonal GLI1 Antibodies

Polyclonal antibodies recognising the GLI1 protein were obtained by immunisation of rabbits by using the GLI1 (1-407) antigen expressed in bacteria by using standard methods. The antibodies obtained were characterised by using the Western blot analysis, Electrophoretic mobility shift assay (EMSA) and immunohistochemical methods.

### EXAMPLE 2

### Obtaining and Characterisation of Polyclonal GLI3 Antibodies

Polyclonal antibodies recognising the GLI3 protein were obtained by immunisation of rabbits by using the GLI3 (150-250) antigen expressed in bacteria and by using standard methods. The antibodies obtained were characterised by using the Western blot analysis, Electrophoretic mobility shift assay (EMSA) and immunohistochemical methods.

### EXAMPLE 3

### Continuation of Peptides Entering Into the Cell to Polyclonal GLI1 Antibodies

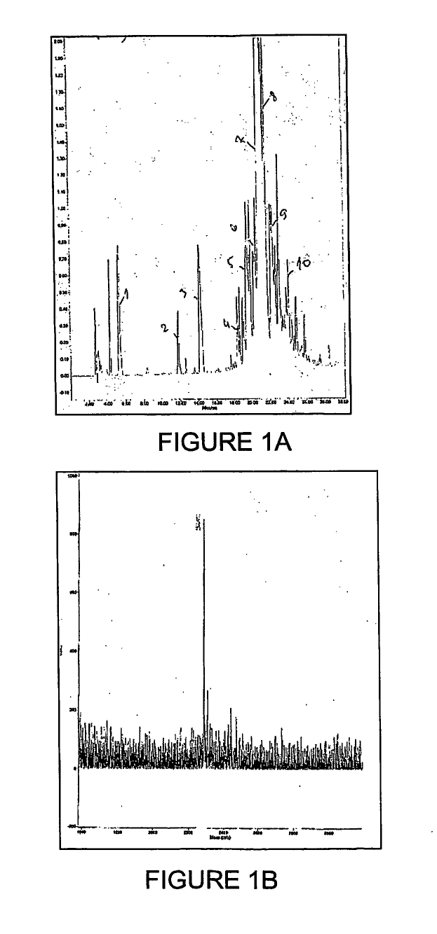

The CPP (Transportan TP10), the shorter analogue of transportan, was synthesised in 0.1 mmol scale on the Applied Biosystem Model 430A peptide synthesizer using the dicyclohexyl carbodimid/hydroxy-benzo-triazole (DCC/HOBT) activation. Peptides were cleaved from the resin according to the TFMSA cleavage protocol. Resulting peptide was further purified on C₁₈ reversed-phase HPLC column that yielded >99% pure product. The molecular mass of each synthetic peptide was determined by MALDI-TOF mass spectrometry and the obtained result was compared with the calculated molecular mass. Transportan 10 (TP10), the shorter analogue of transportan, was conjugated to polyclonal antibodies. FIG. 1 shows the conjugation of cell penetrating peptides to antibodies, which was carried out as follows:

1) CPP was derivatized into maleimid. SMCC solution (succinimidyl 4-(N-maleimidomethyl) cyclohexane-1-carboxylate, Mw 334; 1/5 molar ratio) was added to 200 ml of peptide solution in phosphate buffer (ph 7.5; 10 mg peptide/ml). The mixture described above was incubated for 1-2 hours at room temperature. The SMCC residue was removed by using the HPCL reverse-phase C₁₈ column.

2) In order to deprotect the thiol groups on the antibody, TCEP (tris(2-carboxyethyl)phosphine hydrochloride; Mw 287) in 1/5 molar ratio, was added to the antibody solution (phosphate buffer, pH 7.5) and the reaction mixture was incubated for 15 minutes.

3) Conjugation of maleimid-derivatized peptide to the antibody. Above-mentioned maleimid-derivatized peptide and antibody solution was combined in an equimolar ratio and incubated at room temperature for 3 hours, yielding a thioester bond between the antibody and the peptide. Resulting preparation was used in further experiments, estimating that the conjugative effect was 80%. Peak 7 corresponded to the calculated molecular mass of TP-10 shown on FIG. 1A, as demonstrated by the MALDI-TOF mass spectrometry.

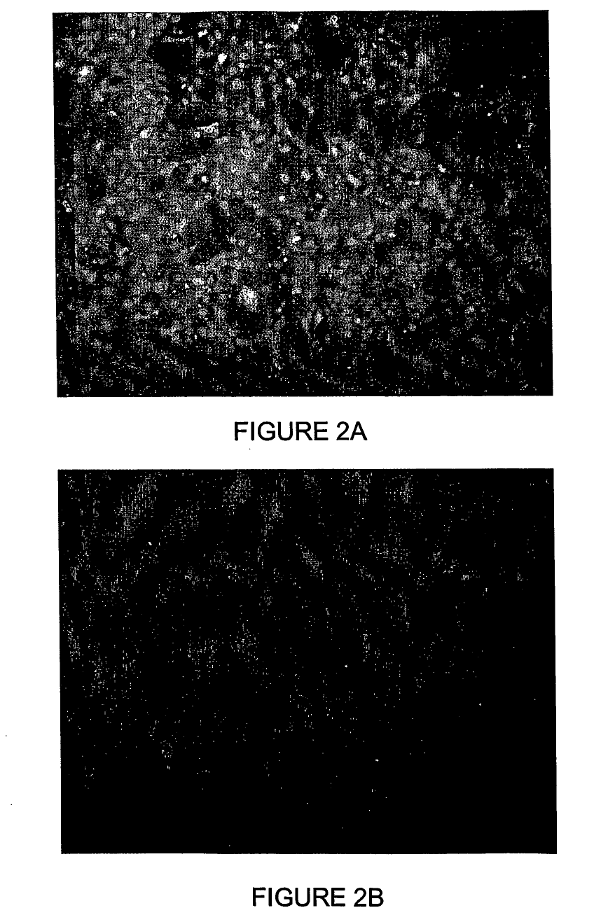

The conjugate obtained was able to enter the eukaryotic cells in culture (FIG. 2). FIG. 2A shows the mouse antiGLI IgG-TP10 conjugate incubated for 3 hours with Cos-1 cells; anti-GLI1 IgG visualised with FITC conjugated mouse anti-IgG antibody. FIG. 2B shows the mouse FITC conjugated anti-IgG, which was incubated for 3 hours with Cos-1 cells. FIG. 2C shows the mouse anti-GLI1 (IgG)-TP10 conjugate incubated for 14 hours with Cos-1 cells; anti-GLI1 IgG visualised with FITC conjugated mouse anti-IgG antibody. FIG. 2D shows the mouse FITC conjugated anti-IgG, which was incubated for 14 hours with Cos-1 cells. The above-mentioned polyclonal antibodies specifically recognised the GLI1 protein.

### EXAMPLE 4

### Obtaining and Characterisation of Anti GLI1 Monoclonal Antibodies

Monoclonal antibodies recognising the GLI1 protein were obtained by immunisation of mice with GLI1 (1-407) protein as an antigen. The protein was expressed in bacteria according to the standard protocol (Antibodies: A Laboratory Manual; Ed. Harlow, David Lane; Cold Spring Harbor Laboratory Press, ISBN: 0879693142). The spleens from immunised mice were dissected and the spleen cells were fused with Sp 2.0 myeloma cells by using standard methods (Antibodies: A Laboratory Manual; Ed. Harlow, David Lane; Cold Spring Harbor Laboratory Press, ISBN: 0879693142). Clones from 40 hybridomas were separated. The resulting antibodies were characterised by Western blot analysis, electromobility shift assay (EMSA) and immunohistochemical methods.

### EXAMPLE 5

### Obtaining and Characterisation of Anti GLI3 Monoclonal Antibodies

Monoclonal antibodies recognising the GLI3 protein were obtained by immunisation of rabbits with GLI3 (150-250) antigen. The protein was expressed in bacteria and according to standard methods (Antibodies: A Laboratory Manual; Ed. Harlow, David Lane; Cold Spring Harbor Laboratory Press, ISBN: 0879693142). The resulting antibodies were characterised by Western blot analysis, electromobility shift assay (EMSA) and immunohistochemical methods.

### EXAMPLE 6

### Developing a Technology for Obtaining Recombinant Cell Penetrating Proteins

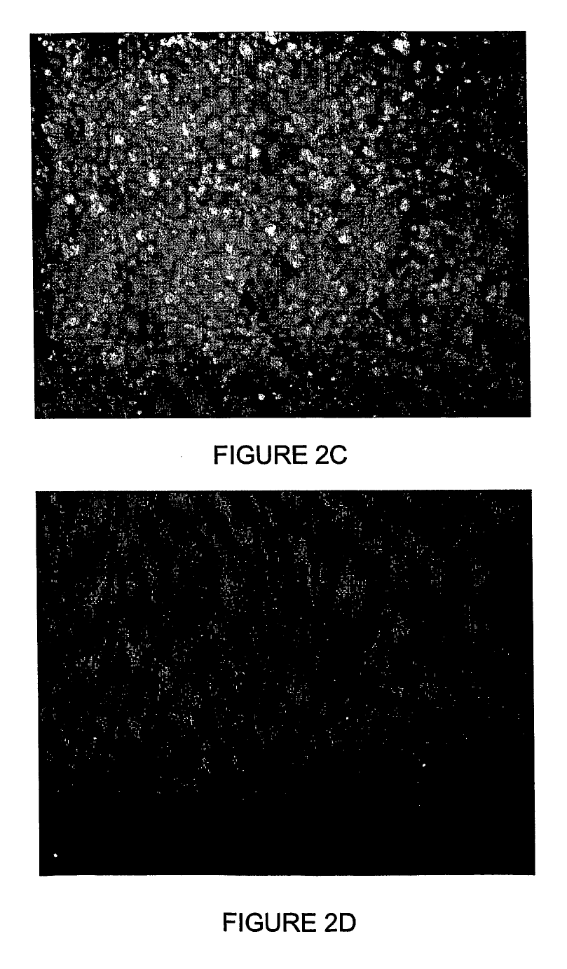

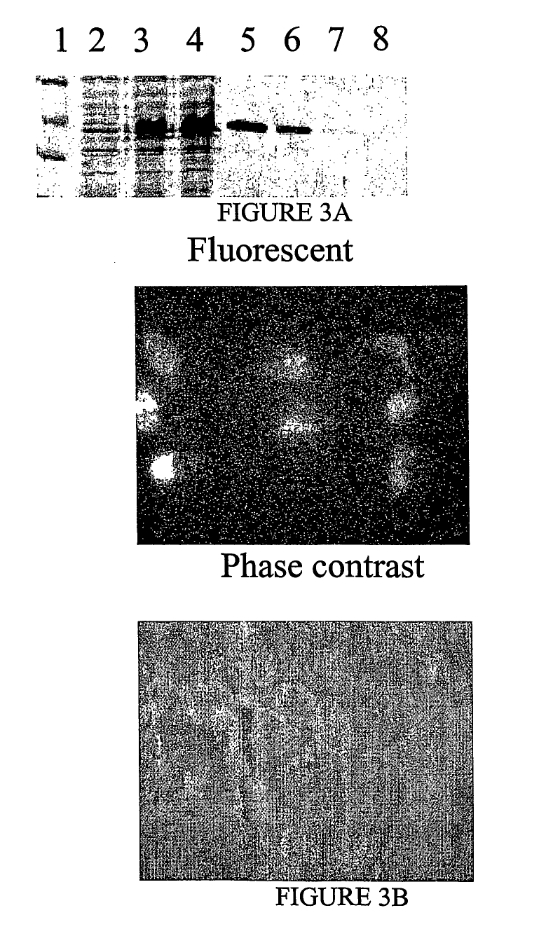

For obtaining a recombinant cell penetrating protein we created expression vector encoding for GST-GLI3 (150-250) fusion protein. We used PCR based approach to add the sequences encoding for cell penetrating peptides Transportan TP10 and 9Arg (9Arginine) into previously mentioned vector. These expression constructs were sequenced. Expression of these constructs showed that despite repeated efforts, it was not possible to express a recombinant fusion protein that encoded GST-GLI3 (150-250)-Transportan TP10 sequence described above in E. Coli system. We succeeded, though, in expressing and purifying a recombinant protein that encoded the recombinant GST-GLI3 (150-250)-9Arg cell penetrating peptide (FIG. 3A).

As we have demonstrated on FIG. 3B, the obtained recombinant protein entered the cultured mammal cells.

### EXAMPLE 7

### Obtaining and Characterisation of Anti GLI Recombinant Proteins Entering Into the Cell

The recombinant antibodies were obtained by inserting the sequence encoding for the 9Arg peptide or Transportan or Transportan TP10 into the gene encoding the clones of antibodies described above. The obtained recombinant antibodies were purified using affinity chromatography and antibody titre was determined. We demonstrated that these antibodies were binding specifically to the GLI1 protein. These recombinant antibodies also entered into the eukaryotic cells in culture.

In order to obtain the scFv with ability to penetrate into the cell we made a construct encoding for single chain antibody, or scFv, containing the two variable domains of an antibody molecule (the VL and the VH domain) linked via flexible peptide linker that also contained the sequence of CPP. The RNAs from the anti GLI1 and 3 monoclonal antibodies were reverse transcribed and this first cDNA strand was used as a template for variable regions amplification using degenerated primers:

T A/C A C C A T G G G A T G G A G A/C T G G A
ATTATCACTGGGTCACTTGAC
TGACAGGCTGGGCTGGCAGGA
A G C/T C T C C C C C/G A/T G G/A G/C C/T T
C T T G C A C A G A/T A A T A C A
GAGCTCGTGATGACCCAGTCTCCA
T T C C A G C T T G G T C/G C C A/G C C A/T
AACACTCATTCCTGTTGAAGC

PCR products of the appropriate size (320-350 bp) were purified and sequenced. Oligonucleotide primer encoding for Transportan or Transportan TP10 and linker (Gly4Ser)₃ was used to construct a VL-TP-Linker-VH sequence by three-step overlap extension PCR. The process was repeated for scFvFc construction with relevant VLCL and VHCH1 PCR products. The final PCR products corresponding scFv and scFvFc (both with CPP and linker encoding) sequence were cloned into eukaryotic expression vector (pcDNA3, pCEP) and sequenced. Eucaryotic cells (Cos-7) were be transfected with scFv or scFvFc constructs and according to the manufacturer's instructions for generation of stable cell lines. Recombinant proteins were purified from supernatant using Ni+ columns.

### References

Dahmane, N., Lee, J., Robins, P., Heller, P., and Ruiz i Altaba, A. (1997). Nature 389, 876-81.

Kogerman, P., Grimm, T., Kogerman, L., Krause, D., Unden, A. B., Sandstedt, B., Toftgard, R, and Zaphiropoulos, P. G. (1999). Nat Cell Biol 1, 312-9.

Lecerf, J. M., Shirley, T. L., Zhu, Q., Kazantsev, A., Amersdorfer, P., Housman, D. E., Messer, A., and Huston, J. S. (2001). Proc Natl Acad Sci USA 98, 4764-9.

Pooga, M., Soomets, U., Hallbrink, M., Valkna, A., Saar, K., Rezaei, K., Kahl, U., Hao, J. X., Xu, X. J., Wiesenfeld-Hallin, Z., Hokfelt, T., Bartfai, T., and Langel, U. (1998). Nat Biotechnol 16, 857-61.

Ridder, R., Schmitz, R., Legay, F., and Gram, H. (1995). Biotechnology (NY) 13, 255-60.

Schwarze, S. R., Ho, A., Vocero-Akbani, A., and Dowdy, S. F. (1999). Science 285, 1569-72.

Steinberger, P., Andris-Widhopf, J., Buhler, B., Torbett, B. E., and Barbas, C. F., 3rd. (2000). Proc Natl Acad Sci USA 97, 805-10.

Strube, R. W., and Chen, S. Y. (2002). J Immunol Methods 263, 149-67.

1-18. (canceled)

19. Fusion protein comprising at least (a) a scFv-part of an antibody, and (b) a cell penetrating transport peptide.

20. Fusion protein comprising at least (a) a scFv-part of an antibody recognising an intracellular GLI-protein, chosen from GLI-1 and GLI-3, and (b) a cell penetrating transport peptide.

21. Fusion protein according to claim 19, wherein the cell-penetrating transport peptide comprises at least a part of Transportan, Transportan 10 or Arg 9.

22. Fusion protein according to any one of claims 19 to 21, wherein the scFv-part is derived from the human genome.

23. Fusion protein according to claim 19, for medical use.

24. Use of the fusion protein according to claim 19, for the preparation of a medicament for treatment of a cancer state.

25. Use according to claim 23, wherein the cancer state is chosen from skin cancer.

26. Pharmaceutical composition comprising at least one fusion protein as defined in claim 19, in association with at least one pharmaceutically acceptable carrier or additive.

27. Method for obtaining a fusion protein as defined in claim 19, wherein the method comprises the steps of expressing the fusion protein and purifying the obtained fusion protein.

28. Method for treatment of a disease or health disorder in humans or animals, comprising administrating a pharmaceutically acceptable dose of the fusion protein as defined in claim 19 to humans or animals.


SEQUENCE LISTING (8 sequences)

SEQ ID NO: 1 - DNA, 23 bp, Mus musculus
tacaccatgggatggagactgga

SEQ ID NO: 2 - DNA, 21 bp, Mus musculus
attatcactgggtcacttgac

SEQ ID NO: 3 - DNA, 21 bp, Mus musculus
tgacaggctgggctggcagga

SEQ ID NO: 4 - DNA, 22 bp, Mus musculus
agctctcccccgatggagcctt

SEQ ID NO: 5 - DNA, 17 bp, Mus musculus
cttgcacagataataca

SEQ ID NO: 6 - DNA, 24 bp, Mus musculus
gagctcgtgatgacccagtctcca

SEQ ID NO: 7 - DNA, 22 bp, Mus musculus
ttccagcttggtcgccagccat

SEQ ID NO: 8 - DNA, 21 bp, Mus musculus
aacactcattcctgttgaagc